(19) United States
(12) Patent Application Publication
(10) Pub. No.: US 2025/0249141 A1
(43) Pub. Date: Aug. 7, 2025

(54) RESIDUAL HAIR FOR TISSUE ENGINEERING AND METHODS OF USING SAME

(71) Applicant: Hofstra University, Hempstead, NY (US)

(72) Inventor: Roche Rey C. de Guzman, Hempstead, NY (US)

(73) Assignee: Hofstra University, Hempstead, NY (US)

(21) Appl. No.: 19/044,445

(22) Filed: Feb. 3, 2025

Related U.S. Application Data
(60) Provisional application No. 63/549,244, filed on Feb. 2, 2024.

(51) Int. Cl. A61L 15/32

(52) U.S. Cl. CPC A61L 15/32; A61L 2300/252; A61L 2300/412

(57) ABSTRACT

The present invention relates to methods of providing residual hair for use in tissue engineering and regenerative medicine (TERM). The method comprises contacting a hair sample with a buffer comprising a reducing agent and alcohol, thereby removing or substantially depleting keratin associated proteins (KAPs), thereby providing residual hair, wherein the residual hair comprises keratins that are organized into intermediate filaments and macrofibrils.

This application claims the benefit of the filing date of U.S. Provisional application 63/549,244, filed on Feb. 2, 2024, the contents of which is incorporated herein by reference in its entirety.

### BACKGROUND OF THE PRESENT INVENTION

Polymeric biomaterials that provide a 3D matrix (e.g., scaffolds and hydrogels/gels) for cell attachment, viability, proliferation, differentiation and functionality while also absorbing and delivering therapeutic agents are important for tissue engineering and regenerative medicine (TERM) strategies. Materials used for such applications typically must have no/minimal toxicity, elicit minimal immune/inflammatory response, have appropriate chemical and mechanical properties such as defined chemistry, high porosity, high water uptake, high surface area-to-volume ratio, and promote favorable cellular responses (allow for cell infiltration, attachment, survival, functionality, and/or proliferation and differentiation). It is generally believed that such materials should degrade in a manner inversely proportional to the rate of tissue formation/regeneration such that the material provides a cell-support matrix but degrades and is cleared by the body to avoid impeding tissue regeneration. These materials are also widely used to achieve controlled release of various exogenous therapeutic and pathogen-inhibitory agents ranging from antibiotics that prevent local infection to small-molecule drugs that penetrate and target cells, and growth factors that promote tissue formation or healing. The two general classes of select polymeric materials used in TERM applications are natural and synthetic polymers.

An example of a natural polymeric material used in TERM applications is human hair. However, there are many disadvantages in the state of the art use of hair for TERM applications. In particular, prior art methods utilize reducing chemistry to obtain only soluble hair extracts, while discarding the hair per se (remaining or residual hair is discarded). The soluble hair extract, often referred to as keratins/keratin proteins/keratin biomaterial(s), is then painstakingly processed to construct hydrogels and/or scaffolds. These hair extract/keratin biomaterial scaffolds/hydrogels from prior art methods are inherently and mechanically weak, degrade very quickly, and appear to elicit slight inflammatory responses in the body. Clearly, more effective methods and compositions are needed for using human hair in TERM applications.

### SUMMARY OF THE PRESENT INVENTION

In one embodiment, the present invention includes methods of providing residual hair for use in tissue engineering and regenerative medicine (TERM). The methods include contacting a hair sample with a buffer comprising a reducing agent and alcohol, thereby removing or substantially depleting keratin associated proteins (KAPs), wherein residual hair is provided. The residual hair comprises keratins that are organized into intermediate filaments and macrofibrils. In some embodiments, at least about 67% (m/m) of the KAPs are removed from the hair sample, thereby leaving less than about 5% (m/m) of KAPs in the hair sample.

In some embodiments, the methods include treating the hair sample with a hydrogen peroxide-based formulation to remove melanin, before the hair sample is contacted with the buffer, thereby providing bleached residual hair.

In some embodiments, the methods further comprise surface degrading the residual hair to remove, or partially remove, the cuticle to produce residual hair with removed or partially removed cuticle. In some embodiments, the methods further comprise surface degrading the bleached residual hair to remove, or partially remove, the cuticle to produce bleached residual hair with removed or partially removed cuticle.

In one embodiment of the present invention, a biomaterial delivery vehicle comprising residual hair is provided. The residual hair comprises keratins that are organized into intermediate filaments and have a macrofibrillar structure, and wherein the residual hair substantially does not comprise KAPs. In some embodiments, at least about 67% (m/m) of the KAPs are removed from the hair sample, thereby leaving less than about 5% (m/m) of KAPs in the hair sample. In some embodiments, the residual hair of the biomaterial delivery vehicle does not comprise melanin. In some embodiments, the biomaterial delivery vehicle comprises a therapeutic agent to be delivered in vivo to a subject in need thereof. In some embodiments, the therapeutic agent comprises a chondromodulin-1 (LECT-1) biomolecule and bone morphogenetic protein-2 (BMP-2) biomolecule, for generating bone and/or cartilage in vivo.

In one embodiment of the present invention, methods of treating open wound are provided. The methods comprise placing a gel on an open wound, wherein the gel comprises residual hair, wherein the residual hair substantially does not comprise KAPs, and substantially do not comprise cuticle, wherein the residual hair comprises keratins that are organized into intermediate filaments and macrofibrils. In some embodiments, at least about 67% (m/m) of the KAPs are removed from the hair sample, thereby leaving less than about 5% (m/m) of KAPs in the hair sample. In some embodiments, the gel is inside an occlusive bandage or dressing. In some embodiments, the open wound is a surgical excision, debridement after burn injury or a diabetic ulcer wound, wherein the epidermis and the dermis of the skin are absent.

### BRIEF DESCRIPTION OF THE DRAWINGS

FIG. 1: Characterization of the Bleached Residual Hair Biomaterial. A) Electron microscopy (scanning and transmission electron microscopy) characterization showing the difference between normal regular/dark hair (REG) and the treated bleached residual hair (BRH). In REG, melanin pigment can be seen as black granules in the bulk hair cortex. Bleaching removes those melanin. Furthermore, the process may remove the outermost cuticle layer (decuticleization), exposing the underlying cortex with organized macrofibrils with high surface area to volume ratio and composed of keratins and keratin associated proteins (KAPs). Finally, KAPs are depleted leaving behind an organized, relatively loose matrix composed of mainly keratin proteins. B) The BRH biomaterial is off white, yellowish gel strands with absorbed water, is easily handleable, and can also be easily placed and delivered via a syringe for injection.

FIG. 2: Proof of Biocompatibility and Degradation of the Bleached Residual Hair Biomaterial. The normal regular hair (REG) induces a relatively thick fibrosis/fibrous encapsulation response, while the treated bleached residual hair (BRH) produced a minimal scar tissue/fibrous capsule formation, indicating biocompatibility of the BRH. From the 2 to 4 week timepoint, BRH is degraded/absorbed by the body as shown in the recovered skin flap after mouse subcutaneous implantation.

FIG. 3: Proof of Bleached Residual Hair Biomaterial as a Functional Delivery Vehicle of Growth Factors for Regeneration of Damaged Tissue, A) A mouse critical-size bone defect is established and treated with the bleached residual hair (BRH) biomaterial loaded with growth factors (GFs) referred to as the BRH+GFs group, and controls: untreated (defect left as is), GFs only (without the biomaterial scaffold), and BRH only (without GFs). B) Computed tomography (CT) imaging reconstruction displaying the control groups: untreated, GFs only, and BRH only did not fully-close the bone gap, while the BRH+GFs group successfully regenerated and closed the defect site at the 8-week endpoint.

FIG. 4: Proof of Functionality of Bleached Residual Hair Biomaterial as a Vehicle of Drugs. Bleached residual hair (BRH) without or with calcium ions (Ca) were loaded with a negatively-charged anti-proliferative drug and the drug's cumulative mass release ratio was evaluated over multiple timepoints in an in vitro assay. The drug was confirmed to be released at a much faster rate in BRH alone due to electrostatic repulsion since BRH is also negatively-charged. Positively-charged calcium ions held the drug as a bridge between two negatively-charged molecules (drug and BRH) leading to a slower drug release profile (left graph), The BRH-released drug was pooled and tested for bioactivity in inhibiting the growth of activated fibroblasts (model for scar tissue). An interpolated concentration from a sigmoidal curve (right graph) at 114 μg/mL led to 50% inhibition (IC50), confirming that the released drug retained its functionality.

FIG. 5: Proof of Concept of Bleached Residual Hair Biomaterial as a Bulk Tissue Filler. The bleached residual hair implant with intact cuticle (right) retained almost its initial form with minimal fibrosis response after 1 month in the subcutaneous region, showing that it can be used as a biocompatible cosmetic implant for increase tissue volume. Comparatively, the polysaccharide biomaterial calcium alginate (left), a relatively nondegradable spherical gel form, is also retained but with high fibrotic tissue response (seen as a white tissue growth).

### DETAILED DESCRIPTION OF THE PRESENT INVENTION

The present invention relates to the process of producing treated hair, including residual hair, for use in applications of tissue engineering and regenerative medicine (TERM), including drug delivery, tissue regeneration and skin wound healing, soft tissue filler, and dermal filler for cosmetics.

Prior art methods utilize reducing chemistry to obtain only soluble extracts from hair. These soluble extracts are often referred to as “keratins” or “keratin proteins” or “keratin biomaterials” but in reality are a mixture composed mostly of keratins, keratin associated proteins (KAPs) and melanin pigments. It has surprisingly been found by the inventor that there are many disadvantages related to KAPs and melanin for use in TERM applications. For example, melanin absorbs light which creates issues in sample imaging; and since melanin's structure is complex, it adds heterogeneity to the biomaterial's composition making future regulatory FDA biologics or medical device premarket approval more difficult. Additionally, melanin's presence complicates protein sample analysis and characterization due to potential binding and interaction with known hair proteins: keratins and KAPs. It has surprisingly been found that oxidation for depletion/removal of melanin per the present invention provides a big advantage for commercialization and clinical translation. The prior art also does not address the surprising disadvantages related to KAPs in human hair-based biomaterials. For example, it has now been found that KAPs elicit fibrous, inflammatory, and immune responses; and identification and characterization using mass spectrometry proteomics is difficult due to less enzymatic cleavage sites and inherent small molecular weight and dozens of uncharacterized heterogenous KAP types in human hair. It has surprisingly been found that the depletion and removal of KAPs from the hair biomaterial decreases effects of fibrosis, inflammation, and the immune response thereby making the biomaterial more biocompatible and more characterized into organized hair keratins. Residual hair is hair that does not contain KAPs or has a minimal level of KAPs. Moreover, by using only the residual hair per the present invention (i.e., hair without KAPs, and without melanin), the manufacturing process for the biomaterial is shortened and the biomaterial has greater strength and organizational properties vis-à-vis prior art biomaterials (i.e., scaffolds/gels formed from hair soluble extracts) due to increased amounts of and/or preserved disulfide crosslinks per mass and macromolecularly-organized keratin proteins structure with intermolecular disulfide, ionic bonds, and hydrophobic interactions. In such a manner, the biomaterial of the present invention is easier to process and handle. Importantly, the biomaterial per the present invention is ready for immediate use compared to prior art extracts that need to be processed further to become hydrogels and scaffolds.

Keratins are a class of intermediate filament cytoskeletal proteins that have received attention for numerous tissue engineering applications because, like other natural polymers, they promote favorable cell interactions, are biocompatible, and have non-toxic degradation products. Keratins can easily be obtained from various sources including sheep wool and human hair, and they are characterized by ability for macromolecular organization and a high percentage of cysteine residues with sulfhydryl or thiol groups. Thiol groups on reductively extracted keratin (also called kerateine) and residual hair form disulfide bonds, providing a means for covalent bonding which includes hydrogel network crosslinking and bonding with added agents. These thiol groups can also be irreversibly oxidized such as exposure to peracetic acid to form sulfonic acid groups that cannot form disulfide crosslinks such as in oxidatively extracted keratin (also called keratose) and residual hair,

### Methods of Making Bleached Residual Hair Biomaterial and Residual Hair Biomaterial

In one embodiment, the invention is the process of obtaining bleached residual hair biomaterial from hair, typically from dark hair, for use in TERM applications. In an example of such a process, in the first step, hair samples are treated with hydrogen peroxide-based formulation to remove melanin and lighten the hair while preserving the hair's keratin and keratin associated proteins (KAPs) and their structural organization. In particular, the hydrogen peroxide-based formulation oxidizes, degrades and removes melanin, while preserving the structural integrity of keratin and KAPs, thereby providing bleached and lightened hair. The bleached hair is then contacted with a buffer containing a reducing agent and alcohol to dissolve and remove the majority of the hair's KAPs, leaving behind mostly keratins that are organized into intermediate filament and macrofibrillar structure in the hair cortex, with or without the outermost cuticle layer. Examples of reducing agents include thioglycolic acid (TGA) and dithiothreitol (DTT). Examples of alcohol include ethanol, isopropanol and 1-butanol. That is, the bleached hair is treated and processed with a buffer containing a reducing agent and alcohol to penetrate the hair cortex and solubilize small protein molecules including KAPs, thereby KAPs are removed from hair after sieving and rinsing, leaving behind the insoluble organized keratins with absorbed water in a hydrogel form, thereby forming bleached residual hair. At least about 67% (m/m) of the KAPs are removed from the hair sample, thereby leaving less than about 5% (m/m) of KAPs in the hair sample. In other embodiments, at least about 70%, 75%, 80%, 85%, 90%, 95% or 99% (m/m) of the KAPs are removed. Preferably all of the KAPs are removed. A buffer is used to stabilize the pH of the residual hair. The resulting bleached residual hair biomaterial comprises (consists essentially of) keratins that are organized into intermediate filaments which are supramolecularly organized into a higher order macrofibrillar structure with water and salt ions in its matrix, without or without cuticle layers.

In an example of one formulation, hair is treated with about 0.3% to 1% (V/V) (typically about 0.6% (V/V)) hydrogen peroxide (H₂O₂) solution, basic pH, at about 10-50 mg hair/mL (typically about 30 mg hair/mL) solution at about 37° C. for about 12-48 hours (typically about 24 hours) with shaking in a closed container, then rinsed with water and strained thrice (3×) to remove the oxidized melanin turning the dark hair, light. After the bleaching process, KAPs are removed/depleted by treating the hair with a solution of about 200-700 mM (typically about 500 mM) thioglycolic acid or dithiothreitol (reducing agent), about 0.1-1.2 M tris base (to adjust the pH to basic), and about 15-40% (V/V) (typically about 25% (V/V)) ethanol (to precipitate and inhibit the solubility of high molecular weight (MW) proteins) at about 10-50 mg hair/mL solution in about 37° C. for about 2-5 days (typically about 3 days) with shaking in a closed container, then rinsed with water and strained thrice (3×) to remove the solubilized KAPs but retaining the organized keratin proteins in the residual hair structure. The residual hair sample is then rinsed with phosphate-buffered saline (PBS) at pH of 7.2-7.4 and strained thrice (3×) to stabilize the pH and make the salt concentration isotonic to living tissues.

In another embodiment, the invention includes a process of obtaining residual hair biomaterial from hair, typically from dark hair, for use in TERM applications. A hair sample is contacted with a buffer containing a reducing agent and alcohol to dissolve and remove the majority of the hair's KAPs, leaving behind mostly keratins that are organized into intermediate filament and macrofibrillar structure in the hair cortex, with or without the outermost cuticle layer. That is, the hair sample is treated and processed with a buffer containing a reducing agent and alcohol to penetrate the hair cortex and solubilize small protein molecules including KAPs, thereby KAPs are removed from hair after sieving and rinsing, leaving behind the insoluble organized keratins with absorbed water in a hydrogel form, thereby forming residual hair. At least about 67% (m/m) of the KAPs are removed from the hair sample, thereby leaving less than about 5t % (m/m) of KAPs in the hair sample. In other embodiments, at least about 70%, 75%, 80%, 85%, 90%, 95% or 99% (m/m) of the KAPs are removed. Preferably all of the KAPs are removed. A buffer is used to stabilize the pH of the residual hair. The resulting residual hair biomaterial comprises (consists essentially of) keratins that are organized into intermediate filaments which are supramolecularly organized into a higher order macrofibrillar structure with water and salt ions in its matrix, with or without intact cuticle layers. This embodiment produces residual hair with preserved melanin pigments.

For example, KAPs are removed/depleted from a hair sample by treating the hair with a solution of about 200-700 mM (typically about 500 mM) thioglycolic acid or dithiothreitol (reducing agent), about 0.1-1.2 M tris base (to adjust the pH to basic), and about 15-40% (V/V) (typically about 25% (V/V)) ethanol (to precipitate and inhibit the solubility of high molecular weight (MW) proteins) at about 10-50 mg hair/mL solution in about 37° C. for about 2-5 days (typically about 3 days) with shaking in a closed container, then rinsed with water and strained thrice (3×) to remove solubilized KAPs but retaining the organized keratin proteins in the residual hair structure. The residual hair sample is then rinsed with phosphate-buffered saline (PBS) at pH of about 7.2-7.4 and strained thrice (3×) to stabilize the pH and make the salt concentration isotonic to living tissues.

In the present invention, a therapeutic/bioactive agent can be incorporated into a residual hair scaffold by covalent and/or non-covalent interactions. Covalent interactions create covalent bonds between the residual hair and the agent, and/or within the scaffold itself. Covalent bonds make residual hair scaffolds less degradable, slows the rate of the activity of agents, and allows for the control of the shape of residual hair scaffolds to better suit a specific target site. Examples of how a covalent interaction can be formed include subjecting and/or exposing the scaffold to a chemical reaction to increase the molecular weight by forming covalent or shared bonds (e.g., exposing a residual hair scaffold to glutaraldehyde), photo (light)-based reaction (e.g., 3D resin printing), thiol-ene reaction, disulfide oxidation reaction, crosslinking reaction using crosslinkers, and combinations thereof. Examples of how a non-covalent interaction can be formed include exposing the residual hair scaffold to physical mixing, dissolution, entanglement, absorption, adsorption, ionic or charged or electrostatic interaction, hydrophobic interaction, 3D filament printing, 3D resin printing, and combinations thereof. The formation of covalent or non-covalent interactions can be made during the formation of the residual hair scaffold and/or after the scaffold has already been formed.

### Biocompatible Scaffolds Comprising Bleached Residual Hair Biomaterial and Residual Hair Biomaterial and Uses Thereof

In one embodiment, the invention provides a biocompatible scaffold comprising bleached residual hair and/or non-bleached residual hair, wherein the residual hair consists of keratins that are organized into intermediate filaments and macrofibrils. The residual hair substantially does not include melanin (at least 179 (>=70%) out of 0-255 8-bit scale where 0=pure black and 255=pure white) and KAPs (at most 5% (<=5%) (m/m) compared to mass of keratins and KAPs; originally, KAPs comprise 15-20% of total mass so bringing it down to <=5% removes at least 67% from the original). Typically, the scaffold is in the form of a hydrogel (scaffold with integrated and absorbed water in its matrix structure). A therapeutic or bioactive agent to be delivered in vivo for tissue engineering and regenerative medicine (TERM) strategies is placed or absorbed into the scaffold.

In specific embodiments, the biocompatible scaffold is employed to absorb drugs and/or biologics, with or without cells, for tissue engineering purposes such as to repair damaged bone and cartilage, inhibit scar tissue formation, or act as a tissue filler. For example, the biocompatible scaffold can be used for: 1) regeneration of damaged tissues by combining with growth factors and/or cells such as bone morphogenetic protein 2 (BMP-2), leukocyte cell-derived chemotaxin-1 (LECT-1), and vascular endothelial growth factor A (VEGF-A), and cultured autologous mesenchymal stem cells (MSCs) and differentiated MSCs into osteoblastic lineage, respectively, 2) drug-delivery vehicle by absorbing small-molecule drugs that can be used for inhibition of fibrous encapsulation or scar tissue such as anti-cell-proliferative drugs including bleomycin and atorvastatin, and for infection prevention such as antibiotics, 3) injected subcutaneously as a cosmetic tissue filler and dermal filler to provide increased local bulk and mass with high retention such as subcutaneous facial fillers for cheeks and lips, and 4) wound healing treatment by adding the residual hair biomaterial, with or without bioactive drugs and growth factors, to full-thickness skin wounds to induce and promote re-epithelialization and tissue healing by activation of local stem cells.

### Process to Remove and Deplete Cuticle

The residual hair's absorption and degradation by the body can be modified through the degree of intactness of the cuticle, the outermost layer of the hair. That is, the more the cuticle is removed, the higher the exposure of the underlying macrofibril and intermediate filament structures in the hair's bulk cortex region, the scaffold degradation rate is increased; whereas, those with intact cuticle degrade at a much slower pace or doesn't degrade at all. Accordingly, residual hair with preserved cuticle is preferred to be used as a cosmetic bulk tissue filler.

For example, the removal/depletion of the cuticle can be effected by the following process. A hair sample is cleaned with soap (such as with 10% sodium dodecyl sulfate (SDS)) and water to remove oil and dirt, then delipidized by treatment of about 67% (V/V) chloroform and about 33% (V/V) methanol to remove the fatty acid layer on the cuticle of mostly 18-methyleicosanonic acid (MEA) to increase hair's hydrophilicity and water penetration. Hair is soaked in about 100% alcohol or acetone and airdried. The degree of decuticleization or removal of cuticle is controlled by time and temperature of hair treatment and exposure to about 5% (m/V) potassium hydroxide (KOH) in 1-butanol. The longer the time and the higher the temperature, the more complete the decuticleization process. For example, in one process, hair at about 0.4% (m/V) in the about 5% KOH in 1-butanol is incubated at about 37-55° C. for about 8-48 hours, followed by stopping the reaction in about 25% (V/V) (hydrochloric acid) HCl in ethanol, then about 100% ethanol, and rinsing in water about five times.

The amount of cuticle in a sample controls how degradable a biomaterial is. The amount of cuticle can be expressed as mass of cuticle per total mass. For example, a sample with fully intact cuticle is 10%=10 g cuticle per 100 g total hair. 1%=1 g cuticle left per 100 g hair, or 90% decuticleized, which is considered a very degradable gel. In some embodiments, the amount of cuticle in the biomaterials of the present invention can be expressed as a percentage of the total cuticle; the amount of cuticle for the biomaterials can range from about 0 to 100%, depending on the specific application.

Residual Hair with Bioactives Used to Regenerate Bone or Cartilage

In one embodiment, the present invention provides methods of generating bone in vivo. In particular, the methods include providing a residual hair biocompatible scaffold containing the bioactives of purified leukocyte cell-derived chemotaxin-1 (LECT-1; also called chondromodulin (ChM-1)) biomolecule, and a bone morphogenetic protein-2 (BMP-2) biomolecule to a subject in need thereof or only LECT-1. The biomolecule can be proteins or nucleic acid molecules (i.e., DNA gene or mRNA) encoding the proteins.

In one embodiment, the methods of the invention include generating cartilage in vivo. The methods include administering a LECT-1 biomolecule to a subject in need thereof. Typically, a LECT-1 molecule is administered without BMP-2 when only cartilage (i.e., not bone) is to be generated.

Subjects who can benefit from the methods of the invention include mammals (e.g., humans and domestic mammals) who are in need of generating bone or cartilage. In some embodiments, the LECT-1, or the LECT-1 and BMP-2, residual hair scaffold, is administered to a subject by injection or surgical implantation into an in vivo target site of the subject in situ. Examples of a target sites in situ for bone regeneration include a bone fracture, bone defect and bone gap. Examples of a target sites in situ for cartilage regeneration include cartilage damage/defect in the articular cartilage of joints, knee meniscus, costal cartilage, intervertebral disc of the spine and ear cartilage.

In other embodiments, instead of administering a residual hair scaffold directly into a target site, the residual hair scaffold is placed outside the cartilages and skeletal system to form cartilage and/or bone ectopically, thereby providing a source for autograft tissues. For example, a scaffold of the present invention is placed into a subcutaneous layer/hypodermis/adipose tissue/subcutaneous white adipose tissue (sWAT)/under the skin, and/or within the muscle tissue for ectopic cartilage formation (i.e., chondrogenesis) or bone formation (i.e., osteogenesis), providing a source of autograft tissues. This autograft tissue, which is a cartilage and/or bone, is then harvested for use in a target cartilage and/or bone defect site.

In embodiments for bone regeneration, the LECT-1 protein can be administered at a range of about 5-125 μg/g target tissue weight; and the BMP-2 protein can be administered at a range of about 1-30 μg/g target tissue weight, when administered in the target region (site); at a BMP-2-to-LECT-1 molecule count ratio of about 100-2600:1. As would be known to a skilled artisan, for the administration of nucleic acids, dosing would depend on the specific features of the DNA/RNA vector used, including the specific promoters and other construct components. For example, for LECT-1 DNA/RNA, the dose can range from about 10 to 50 μg/g target tissue weight; and for BMP-2 DNA/RNA the dose can range from about 0.5 to 12.5 mg/g target tissue weight.

In embodiments for cartilage regeneration, the LECT-1 protein can be placed into the residual hair scaffold at a range of about 100-2500 μg/g target tissue weight; and the BMP-2 protein can be placed into the residual hair scaffold at a range of about 0-4 μg/g target tissue weight, when administered in the target region (site); at a BMP-2-to-LECT-1 molecule count ratio of about 0-16:1. As would be known to a skilled artisan, for the administration of nucleic acids, dosing would depend on the specific features of the DNA/RNA vector used, including the specific promoters and other construct components. For example, for LECT-1 DNA/RNA, the dose can range from about 200 to 1000 μg/g target tissue weight; and for BMP-2 DNA/RNA the dose can range from about 0 to 2 mg/g target tissue weight.

In one embodiment, the LECT-1 protein can be placed into the residual hair scaffold at a range of about 1-50 μg/kg body weight; and the BMP-2 protein can be placed into the residual hair scaffold at a range of about 100-600 μg/kg, when administered in the target region. In one embodiment, LECT-1 DNA/RNA can be administered in a dose range from about 0.5 to 20 μg/kg body weight; and BMP-2 DNA/RNA can be administered in a dose range from about 25 to 300 μg/kg body weight.

### Residual Hair Biomaterial as a Wound Healing Treatment

In one embodiment, the residual hair biomaterial of the present invention can be used to treat wounds, e.g., used as a thick gel inside an occlusive bandage or dressing (e.g., a hydrogel). For example, residual hair with removed cuticle (or degradable form) can be applied on an open wound caused by surgical excision/debridement after burn injury or in a diabetic ulcer wound where the epidermis and the dermis of the skin are removed (complete thickness).

The skin's outermost layer is the epidermis which is a tissue composed of cells called the keratinocytes and a flat extracellular matrix (ECM), the basal lamina or basement membrane (BM). Directly attached on the BM are adult stem cells of the epidermis, the epidermal stem cells (ESCs). ESCs are the source of keratinocytes through the biological processes of activation, migration, proliferation, and differentiation. The main cytoskeletal proteins of keratinocytes are keratins/cytokeratins which are also known to activate ESCs, and are suggested to be more bioactive in organized keratin. Accordingly, since hair keratins and cytokeratins are keratin proteins, the addition of organized version (residual hair) to skin wounds would activate ESCs leading to re-epithelization which seals off the wound, protecting it from complications including bacterial infection and water loss. Additionally, mesenchymal stem cells (MSCs) and fibroblasts (progenitor cells), the cells responsible for replenishing the skin's second and thickest layer, the dermis, are activated by residual hair to be primed for regeneration. Hence, the residual hair biomaterial can repair and regenerate skin wounds via the activation of the body's skin stem cells (ESCs and MSCs) and progenitor cells in adjacent intact skin, to ultimately differentiate into functional skin cells: keratinocytes and fibroblasts and fibrocytes, which in turn secretes their respective ECM: BM and 3D fiber matrices during the final remodeling and maturation of the wound healing processes.

### EXAMPLES

### Characterization of Bleached Residual Hair Biomaterial

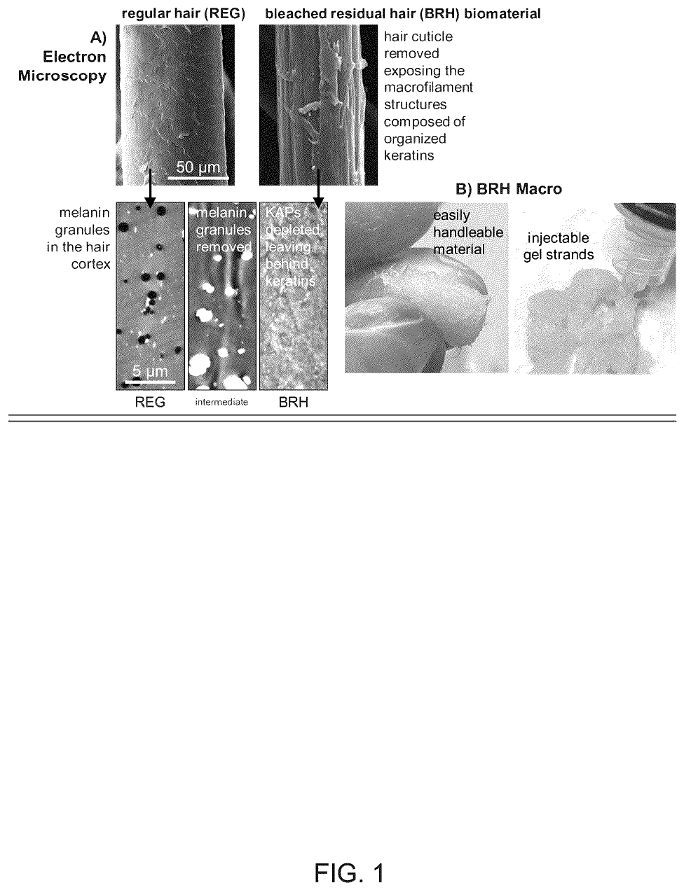

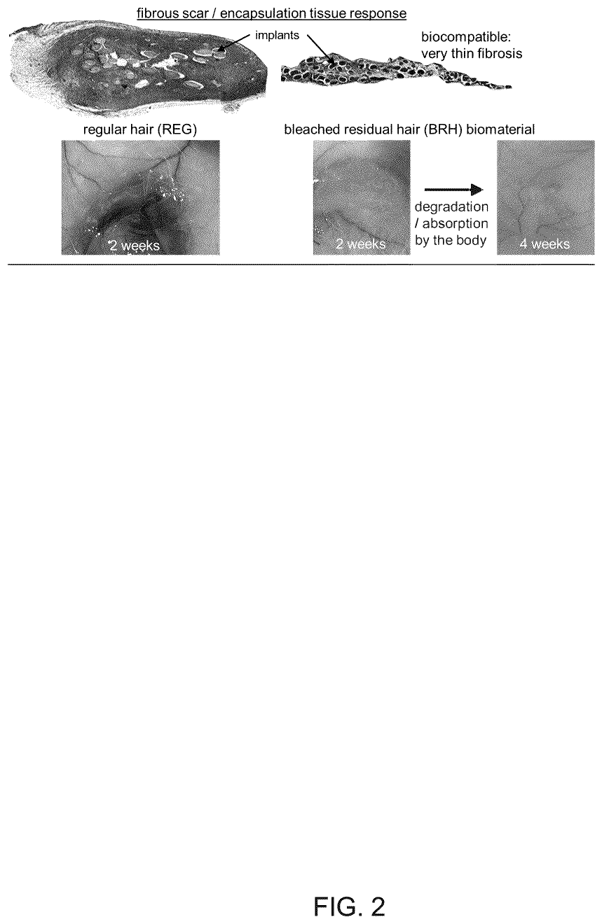

The bleached residual hair biomaterial of the present invention has been characterized for absence or minimal melanin and KAPs content compared to a regular hair (FIG. 1). In particular, hair samples are bleached to dissolve melanin, then soluble small-molecule keratin associated proteins (KAPs) are also removed/depleted while keeping the hair's fibrous structures and assembled keratins intact using controlled treatment of reducing agents and alcohol. The bleached residual hair biomaterial appears off white, yellowish, translucent composed of hydrogel/gel strands with absorbed water and high surface area to volume ratio. It has been tested for both in vitro and in vivo biocompatibility (FIG. 2) and bioactivities with excellent outcomes.

### Bleached Residual Hair Biomaterial Used in Tissue Regeneration

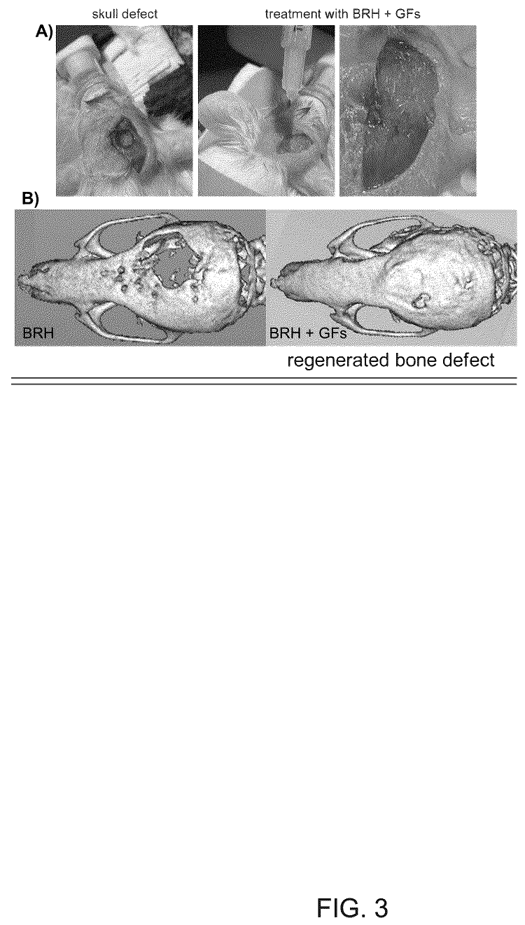

In tissue regeneration using scaffold-delivered growth factors (GFs), it was found that the bleached residual hair is an effective and functional GF-delivery vehicle/gel (FIG. 3). Specifically in a mouse critical-size skull defect model, the gap left untreated, as well as treatment with only the GFs or the scaffold (without GFs) resulted in retention of the bone defect; indicating fast GF clearance when there is no vehicle scaffold and no regenerative activity of the scaffold only, respectively. However, treatment with the bleached residual hair plus GFs led to full gap closure and successful bone regeneration.

### Bleached Residual Hair Biomaterial as a Drug-Delivery Vehicle

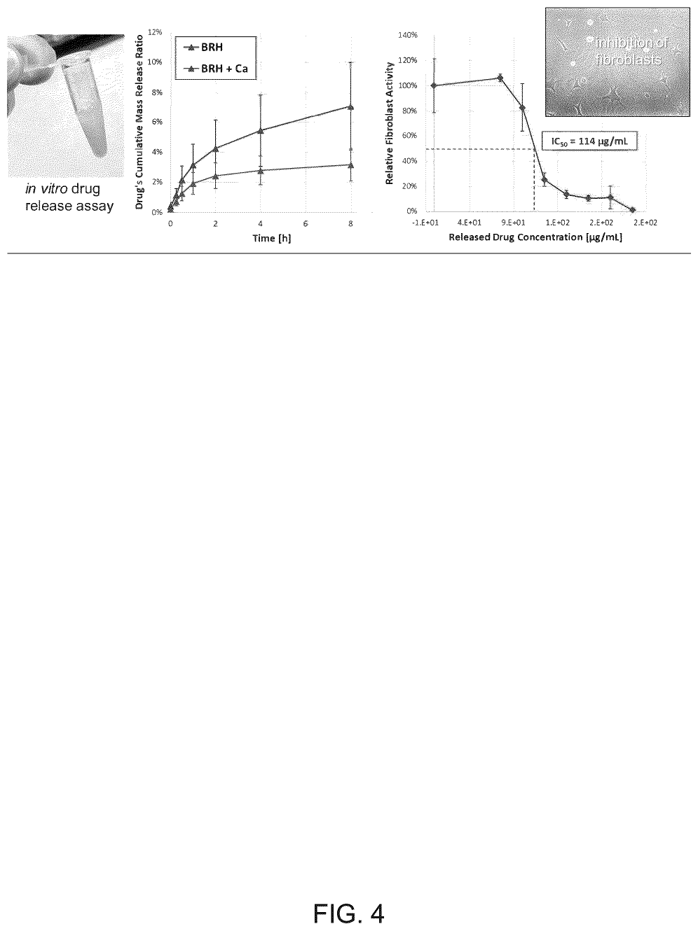

As a drug-delivery vehicle, the bleached residual hair was shown to absorb/sequester small-molecule pharmaceutical drugs, then subsequently release in an in vitro model (FIG. 4). The released drug was shown to be functional specifically in inhibiting the proliferation of activated fibroblasts or scar tissues.

### Bleached Residual Hair Biomaterial as a Cosmetic Bulk Tissue Filler

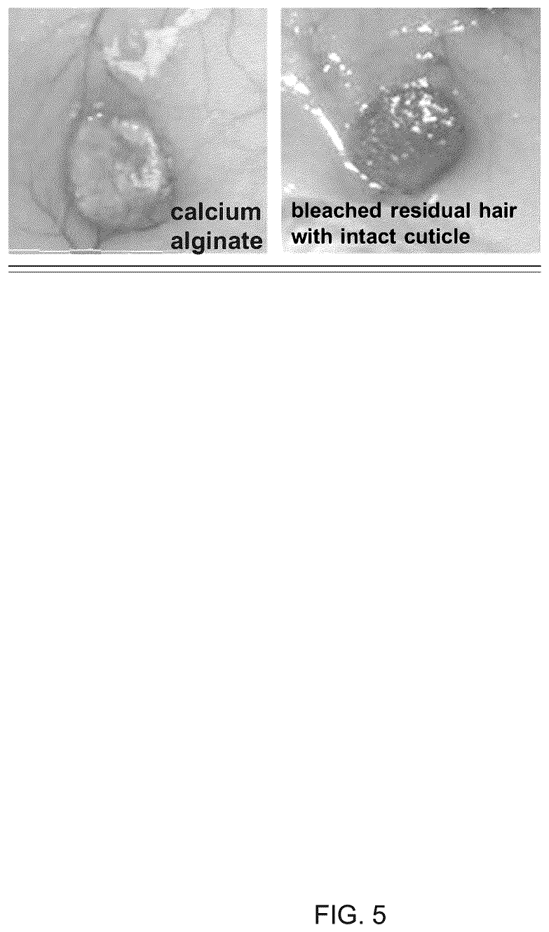

One method to control the bleached residual hair's absorption and degradation by the body is through the cuticle layer. Samples with removed cuticle exposing the underlying macrofibril structures in the hair's bulk cortex region have faster degradation rate due to relatively less disulfide bonding, while those with intact cuticle degrade at a much slower pace or even with no significant degradation over time, since cuticular keratins are known to contain denser amount of covalent and strong disulfide bridges. Accordingly, bleached residual hair with preserved or intact cuticle can be used as a cosmetic bulk tissue and/or dermal filler (FIG. 5).

As various changes may be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

1. A method of providing residual hair for use in tissue engineering and regenerative medicine (TERM), the method comprising:
contacting a hair sample with a buffer comprising a reducing agent and alcohol, thereby removing or substantially depleting keratin associated proteins (KAPs),
thereby providing residual hair, wherein the residual hair comprises keratins that are organized into intermediate filaments and macrofibrils.

2. The method of claim 1, wherein at least about 67% (m/m) of the KAPs are removed from the hair sample, thereby leaving less than about 5% (m/m) of KAPs in the hair sample.

3. The method of claim 1, further comprising treating the hair sample with a hydrogen peroxide-based formulation to remove melanin, before the hair sample is contacted with the buffer, thereby providing bleached residual hair.

4. The method of claim 1, further comprising surface degrading the residual hair to remove, or partially remove, the cuticle to produce residual hair with removed or partially removed cuticle.

5. The method of claim 3, further comprising surface degrading the bleached residual hair to remove, or partially remove, the cuticle to produce bleached residual hair with removed or partially removed cuticle.

6. A biomaterial delivery vehicle comprising residual hair, wherein the residual hair comprises keratins that are organized into intermediate filaments and have a macrofibrillar structure, and wherein the residual hair substantially does not comprise KAPs.

7. The biomaterial delivery vehicle of claim 6, wherein at least about 67% (m/m) of the KAPs are removed from the hair sample, thereby leaving less than about 5% (m/m) of KAPs in the hair sample.

8. The biomaterial delivery vehicle of claim 6, wherein the residual hair further does not comprise melanin.

9. The biomaterial delivery vehicle of claim 6, wherein the vehicle comprises a therapeutic agent to be delivered in vivo to a subject in need thereof.

10. The biomaterial delivery vehicle of claim 9, wherein the therapeutic agent comprises a chondromodulin-1 (LECT-1) biomolecule and bone morphogenetic protein-2 (BMP-2) biomolecule, for generating bone and/or cartilage in vivo.

11. A method of treating an open wound comprising:
placing a gel on an open wound,
wherein the gel comprises residual hair, wherein the residual hair substantially does not comprise KAPs, and substantially do not comprise cuticle,
wherein the residual hair comprises keratins that are organized into intermediate filaments and macrofibrils.

12. The method of claim 11, wherein at least about 67% (m/m) of the KAPs are removed from the hair sample, thereby leaving less than about 5% (m/m) of KAPs in the hair sample.

13. The method of claim 12, wherein the gel is inside an occlusive bandage or dressing.

14. The method of claim 13, wherein the open wound is a surgical excision, debridement after burn injury or a diabetic ulcer wound, wherein the epidermis and the dermis of the skin are absent.
